(19) United States
(12) Patent Application Publication
(10) Pub. No.: US 2024/0221905 A1
(43) Pub. Date: Jul. 4, 2024

(54) PROCESSING APPARATUS, PROCESSING METHOD, PROCESSING PROGRAM, AND STORAGE SYSTEM

(71) Applicant: TOYOTA JIDOSHA KABUSHIKI KAISHA, Toyota-shi (JP)

(72) Inventors: Issei NAKASHIMA, Katsushika-ku (JP); Toru MIYAGAWA, Seto-shi (JP); Hideo HASEGAWA, Nagoya-shi (JP); Yusuke KINOSHITA, Ota-ku (JP); Shotaro YASUDA, Taito-ku (JP); Narimasa WATANABE, Chofu-shi (JP); Mika SAKODA, Setagaya-ku (JP); Saki HAYAKAWA, Edogawa-ku (JP)

(73) Assignee: TOYOTA JIDOSHA KABUSHIKI KAISHA, Toyota-shi (JP)

(21) Appl. No.: 18/402,092

(22) Filed: Jan. 2, 2024

(30) Foreign Application Priority Data
Jan. 4, 2023 (JP) 2023-000139

(51) Int. Cl. G16H 20/60; G07C 9/00

(52) U.S. Cl. CPC G16H 20/60; G07C 9/00896

(57) ABSTRACT

A processing apparatus that can encourage a user to pick up a food item that is preferably consumed is achieved. A processing apparatus according to an example aspect of the present disclosure includes a data acquisition unit configured to acquire lifestyle data and personal data of a consumption requester of food items and a control unit configured to generate recommendation data for food items to be recommended for the consumption requester to consume based on the lifestyle data and the personal data, and control a storage unit so that the recommended food items stored in the storage unit becomes accessible by the consumption requester based on the recommendation data.

### CROSS REFERENCE TO RELATED APPLICATIONS

This application is based upon and claims the benefit of priority from Japanese patent application No. 2023-000139, filed on Jan. 4, 2023, the disclosure of which is incorporated herein in its entirety by reference.

### BACKGROUND

The present disclosure relates to a processing apparatus, a processing method, a processing program, and a storage system.

It is preferable for the sake of health to consume a balanced diet of healthy meals and snacks, and there are various techniques for proposing such a balanced diet of healthy meals and snacks. For example, a food information provision service system described in Japanese Unexamined Patent Application Publication No. 2002-366663 generates data regarding food items which would be preferable for a user to purchase and then consume based on user data including food cost data related to the user's meals and nutritional data on food items, and then suggests them to the user.

### SUMMARY

The applicant has found out the following problem with the above technique. For instance, when items such as snacks and other favorite products are on shelves, people tend to unconsciously reach for the favorite products. Therefore, merely suggesting to a user certain food items are preferable for consumption, as in the food information provision service described in Japanese Unexamined Patent Application Publication No. 2002-366663, may not necessarily lead the user to actually pick up those food items.

The present disclosure has been made to solve such a problem and provides a processing apparatus, a processing method, a processing program, and a storage system that can encourage a user to pick up a food item that is preferably consumed.

A processing apparatus according to one example aspect of the present disclosure includes:
a data acquisition unit configured to acquire lifestyle data and personal data of a consumption requester of food items; and
a control unit configured to generate recommendation data for food items to be recommended for the consumption requester to consume based on the lifestyle data and the personal data, and control a storage unit so that the recommended food items stored in the storage unit becomes accessible by the consumption requester based on the recommendation data.

In the above processing apparatus, the control unit preferably generates the recommendation data based on data indicating a difference between an upper limit calorie intake of the consumption requester over a preset period calculated from the personal data and consumed calories over the preset period calculated from the lifestyle data.

In the above processing apparatus, the data acquisition unit preferably acquires health data of the consumption requester as the lifestyle data, and
the control unit preferably generates the recommendation data based on data indicating a blood sugar level or a stress state of the consumption requester acquired from the health data.

In the above processing apparatus, the data acquisition unit preferably acquires schedule data of the consumption requester as the lifestyle data, and
the control unit preferably generates the recommendation data based on data indicating a snack time estimated from the schedule data.

In the above processing apparatus, the control unit preferably controls the storage unit to unlock a locked state of an area containing the recommended food items among a plurality of locked areas containing food items within the storage unit based on the recommendation data.

In the above processing apparatus, the control unit preferably generates non-recommendation data for food items that are not recommended to be consumed by the consumption requester based on the personal data, and controls the storage unit based on non-recommendation data such that the food items that are not recommended are arranged in preset hard-to-reach areas within the storage unit.

In the above processing apparatus, a preset accessible area for food items within the storage unit is arranged on a front side of the storage unit in relation to the hard-to-reach area.

In the above processing apparatus, the preset hard-to-reach area for the food items within the storage unit is arranged in a position of the storage unit that is higher or lower than that of the accessible area for the food items.

A storage system according to an example aspect of the present disclosure includes:
the above processing apparatus; and
a storage unit containing the food items.

A processing method according to an example aspect of the present disclosure includes:
acquiring lifestyle data and personal data of a consumption requester of food items; and
generating recommendation data for food items to be recommended for the consumption requester to consume based on the lifestyle data and the personal data, and controlling a storage unit so that the recommended food items stored in the storage unit becomes accessible by the consumption requester based on the recommendation data.

A processing program according to an example aspect of the present disclosure causes a computer to execute processing of:
acquiring lifestyle data and personal data of a consumption requester of food items; and
generating recommendation data for food items to be recommended for the consumption requester to consume based on the lifestyle data and the personal data, and controlling a storage unit so that the recommended food items stored in the storage unit becomes accessible by the consumption requester based on the recommendation data.

According to the present disclosure, it is possible to achieve a processing apparatus, a processing method, a processing program, and a storage system that can encourage a user to pick up a food item that is preferably consumed.

The above and other objects, features and advantages of the present disclosure will become more fully understood from the detailed description given hereinbelow and the accompanying drawings which are given by way of illustration only, and thus are not to be considered as limiting the present disclosure.

### BRIEF DESCRIPTION OF DRAWINGS

FIG. 1 is a block diagram showing a configuration of a control system of a storage system according to a first embodiment;

FIG. 2 is a front view showing a storage unit main body and boxes of a storage unit according to the first embodiment in a simplified manner;

FIG. 3 is a flowchart diagram showing an example of a flow of arranging the box in an area of the storage unit main body using a processing apparatus according to the first embodiment;

FIG. 4 is a block diagram showing a control system of a storage system according to a second embodiment;

FIG. 5 is a block diagram showing a control system of a storage system according to a third embodiment;

FIG. 6 is a block diagram showing a control system of a storage system according to a fourth embodiment; and

FIG. 7 is a front view showing a storage unit main body and boxes of a storage unit according to a fourth embodiment in a simplified manner.

### DESCRIPTION OF EMBODIMENTS

Specific embodiments to which the present disclosure is applied will now be described in detail with reference to the drawings. However, the present disclosure is not limited to the following embodiments. The following description and drawings are simplified as appropriate.

### First Embodiment

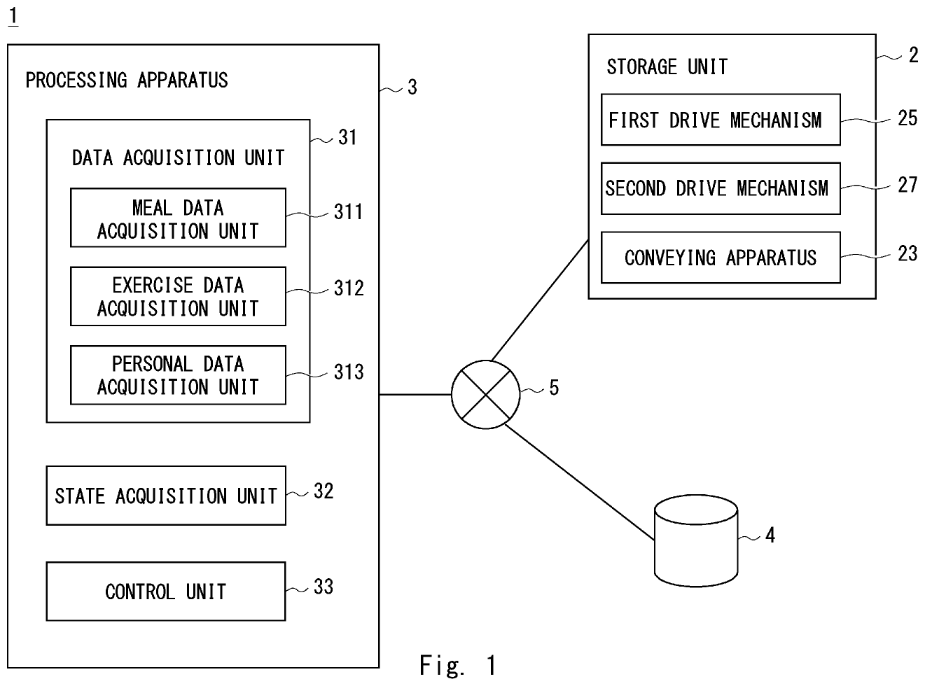

FIG. 1 is a block diagram showing a configuration of a control system of a storage system according to this embodiment. As shown in FIG. 1, a storage system 1 includes a storage unit 2, a processing apparatus 3, and a database 4. The storage unit 2, the processing apparatus 3, and the database 4 are connected through a network 5. The network 5 is a wired or wireless communication line, for example, the Internet.

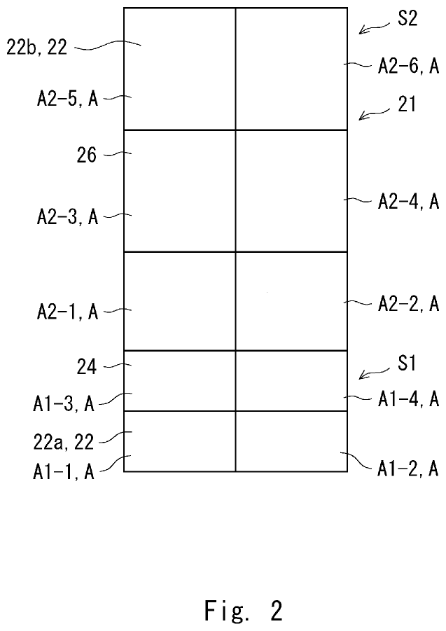

FIG. 2 is a front view showing a storage unit main body and boxes of a storage unit according to this embodiment in a simplified manner. As shown in FIGS. 1 and 2, the storage unit 2 includes a storage unit main body 21, boxes 22, and a conveying apparatus 23. The storage unit main body 21 is, for example, a fridge-freezer divided into a plurality of rectangular areas A as viewed from the front and rear directions, and the front side (i.e., front face) of the storage unit main body 21 is arranged on the side of a living area.

The storage unit main body 21 includes, for example, a freezer space S1 and a refrigeration space S2 as shown in FIG. 2. The freezer space S1 includes, for example, the following areas when viewed from the front of the storage unit main body 21: a first area A1-1 in the lower left, a second area A1-2 in the lower right, a third area A1-3 in the upper left, and a fourth area A1-4 in the upper right. Each of these areas A1-1, A1-2, A1-3, and A1-4 is temperature-controlled to maintain the frozen state of food items.

At this point, for instance, based on the user of the storage unit 2, who is an individual requesting to consume food items, and considering his/her physique, the first area A1-1 and the second area A1-2 are designated as areas that are not easily accessible (e.g., hard-to-reach areas), while the third area A1-3 and the fourth area A1-4 are designated as easily accessible areas (e.g., accessible areas).

In other words, the hard-to-reach areas are positioned lower within the storage unit main body 21 in comparison to the accessible areas. It is preferable that each of the areas A1-1, A1-2, A1-3, and A1-4 have a substantially equal shape.

For example, as shown in FIG. 2, the refrigeration space S2 is arranged above the freezer space S1. The refrigeration space S2 includes, for example, the following areas when viewed from the front of the storage unit main body 21: a first area A2-1 in the lower left, a second area A2-2 in the lower right, a third area A2-3 in the middle left, a fourth area A2-4 in the middle right, a fifth area A2-5 in the upper left, and a sixth area A2-6 in the upper right. Each of these areas A2-1, A2-2, A2-3, A2-4, A2-5, A2-6 is temperature-controlled to maintain the refrigerated state of food items.

At this time, for example, based on the user's physique, the first area A2-1, the second area 2-2, the fifth area A2-5, and the sixth area A2-6 are hard-to-reach areas, and the third area A2-3 and the fourth area A2-4 are accessible areas.

That is, the hard-to-reach areas are arranged at a position within the storage unit main body 21 that is higher or lower than that of the accessible areas. Here, it is preferable that each of the areas A2-1, A2-2, A2-3, A2-4, A2-5, and A2-6 have a substantially equal shape.

As shown in FIGS. 1 and 2, a first door 24 that can be opened and closed is provided on the front and rear sides of the freezer space S1, and opening and closing of the first door 24 on at least the rear side of the freezer space S1 is controlled by a first drive mechanism 25.

Further, as shown in FIGS. 1 and 2, a second door 26 that can be opened and closed is provided on the front and rear sides of the refrigeration space S2, and opening and closing of the second door 26 on at least the rear side of the refrigeration space S2 is controlled by a second drive mechanism 27.

The boxes 22 are designed to be capable of storing food items inside, and they have shapes that can be stored within the areas A of the storage unit main body 21. Here, the food items include not only food but also beverages and may be consumed by human beings through their mouths.

For example, as shown in FIG. 2, two types of boxes 22 may be provided: a first box 22a, which can be carried in and taken out (inserted and removed) from the front and rear sides of the storage unit main body 21 in each of the areas A1-1, A1-2, A1-3, and A1-4 of the freezer space S1, and a second box 22b, which can be carried in and taken out from the front and rear sides of the storage unit main body 21 in each of the areas A2-1, A2-2, A2-3, A2-4, A2-5, and A2-6 of the refrigeration space S2.

The conveying apparatus 23 carries the boxes 22 into the areas A of the storage unit main body 21, carries the boxes 22 out of the areas A of the storage unit main body 21, and exchanges the boxes 22 between different areas A of the storage unit main body 21. That is, the conveying apparatus 23 inserts the first box 22a into each of the areas A1-1, A1-2, A1-3, and A1-4 of the freezer space S1 and removes it from each of the areas A1-1, A1-2, A1-3, and A1-4.

The conveying apparatus 23 inserts the second box 22b into each of the areas A2-1, A2-2, A2-3, A2-4, A2-5, and A2-6 of the refrigeration space S2 and removes it from each of the areas A2-1, A2-2, A2-3, A2-4, A2-5, and A2-6.

The conveying apparatus 23 can be composed of a robot, for example, which generally conveys a box or the like to a shelf in order to achieve the above-mentioned actions. Here, the conveying apparatus 23 may be, for example, configured to be movable.

As shown in FIG. 1, the processing apparatus 3 includes a data acquisition unit 31, a state acquisition unit 32, and a control unit 33. The processing apparatus 3 can be implemented by one or more computers. The processing apparatus 3 is typically an information processing apparatus such as a server apparatus.

Specifically, the processing apparatus 3 includes a memory for storing programs and the like, and a processor for executing the programs. The processing apparatus 3 may include a plurality of processors for performing distributed processing. For example, a server apparatus and a user terminal connected by the network 5 may cooperate to perform processing.

The data acquisition unit 31 acquires data related to the user. As shown in FIG. 1, the data acquisition unit 31 includes a meal data acquisition unit 311, an exercise data acquisition unit 312, and a personal data acquisition unit 313. The meal data acquisition unit 311 acquires meal data related to meals consumed by the user, which is an example of lifestyle data.

The meal data is, for example, data indicating a meal menu and a mealtime. The meal data acquisition unit 311 acquires data related to nutrients, ingredients, food items, and the like from the meal menu that the user has consumed. The meal data includes not only breakfast, lunch, and dinner, but also snacks and late-night snacks.

The meal data includes, for example, data showing the food items in the meal and their amounts. The meal data also include, for example, information related to calorie intake, sugar content, salt content, fat content, vitamins, carbohydrates, and other nutrients. In addition, the meal data includes data on mealtimes at which the user consumed meals.

In other words, the meal data includes the mealtimes, nutrients, meal menus associated with each other. The meal data acquisition unit 311 stores time-series meal data in the database 4 or the like.

The meal data can be acquired from image data captured of the user's meals. The meal data acquisition unit 311 may acquire the meal data using a sensor for detecting the meal data. Alternatively, the user or other parties may input the consumed meal menus, so that the meal data acquisition unit 311 can acquire the meal data.

The exercise data acquisition unit 312 acquires exercise data related to the user's exercise, which is an example of the lifestyle data. Here, the exercise includes not only activities such as walking, jogging, cycling, and using a foot-pedaling machine, but also includes any action by the user that results in calorie consumption.

The exercise data is, for example, data indicating the exercise performed by the user and exercise time. The exercise data includes, for example, data such as a heart rate, a body temperature, and an exercise volume. In other words, in the exercise data, the exercise, the exercise time, and the exercise volume are associated with each other.

The user may input his/her performed exercise so that the exercise data acquisition unit 312 can acquire the exercise data. Alternatively, a wearable device such as a smartwatch may be used so that the exercise data acquisition unit 312 can detect the user's exercise.

Further alternatively, a camera may be used to detect the user's exercise. In other words, the exercise data acquisition unit 312 may utilize a sensor to detect the exercise. The exercise data acquisition unit 312 records the time-series exercise data in the database 4 or the like.

The personal data acquisition unit 313 acquires personal data related to the user. For example, the personal data acquisition unit 313 acquires personal data related to, for example, the user's height, weight, age, and gender.

The personal data may also include, for example, data related to the user's body fat percentage, body fat amount, and muscle mass. Further, the personal data may also include data related to the user's occupation and medical history. The personal data acquisition unit 313 stores this personal data in the database 4 or the like.

The state acquisition unit 32 calculates the consumed calorie for the user within a preset period based on the exercise data and then generates consumed calorie data. The state acquisition unit 32 can calculate the consumed calorie by, for example, a general consumed calorie calculation method. The state acquisition unit 32 stores the consumed calorie data in the database 4 or the like.

Furthermore, the state acquisition unit 32 calculates an upper limit calorie intake for the user within a preset period based on the personal data and then generates the upper limit calorie intake data. The state acquisition unit 32 can calculate the user's upper limit calorie intake using a preset upper limit calorie intake tailored by considerations of factors such as height, weight, age, and gender. In this process, the state acquisition unit 32 may also consider the lifestyle data in accordance with the preset period to calculate the upper limit calorie intake.

When the personal data includes information about the user's body fat percentage and the like, the state acquisition unit 32 may consider this data to calculate the upper limit calorie intake. Additionally, if the user's schedule data regarding planned exercise can be acquired, the state acquisition unit 32 may also consider this data to calculate the upper limit calorie intake. The state acquisition unit 32 stores the upper limit calorie intake data in the database 4 or the like.

The control unit 33 generates recommendation data for food items to be recommended for the user to consume based on the lifestyle data and the personal data. Based on this recommendation data, the control unit 33 controls the storage unit 2 to ensure that the recommended food items stored within it become accessible to the user.

For example, although the details will be described later, the control unit 33 calculates a difference between the upper limit calorie intake and the consumed calorie, and controls either the first drive mechanism 25 or the second drive mechanism 27 and also controls the conveying apparatus 23 so that the food items with calories corresponding to this difference are arranged (stored) in the accessible area.

The database 4 stores the meal data, the exercise data, the personal data, arrangement data of the boxes 22, data related to the food items stored in the boxes 22, and the like. At this time, data related to the food items stored in the boxes 22 is stored in association with the arrangement data of the boxes 22, and when the boxes 22 are delivered by a delivery service, for example, the data related to the food items stored in the boxes 22 can be acquired based on delivery data from the delivery service and the like.

In addition, the database 4 stores food item data of each item such as calorie, sugar content, salt content, fat content, vitamins, carbohydrates, and other nutrients for each preset food item. In this context, the database 4 may store ranking data for recommending food items to the user based on each item of the food item data.

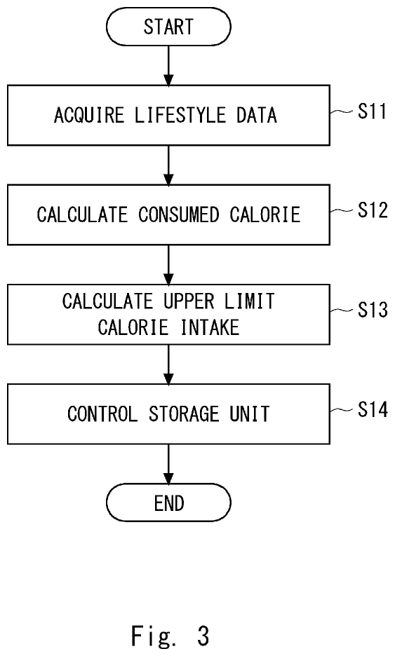

Next, an example of a flow of arranging the boxes 22 in the areas A of the storage unit main body 21 using the processing apparatus according to this embodiment will be described. FIG. 3 is a flowchart showing an example of the flow of arranging the boxes in the areas of the storage unit main body using the processing apparatus according to this embodiment.

Here, in this embodiment, the focus is on recommending food items to the user based on calories, but a similar approach can also be applied when recommending food items based on factors such as salt content, fat content, vitamins, and other nutrients. It is assumed that the personal data acquisition unit 313 of the processing apparatus 3 acquires the personal data related to the user in advance and stores the personal data in the database 4.

First, the data acquisition unit 31 of the processing apparatus 3 acquires the user's meal data and exercise data, that is, lifestyle data, and stores the meal data and exercise data in the database 4 or the like (S11). Next, the state acquisition unit 32 of the processing apparatus 3 calculates the user's consumed calorie within a preset period based on the exercise data, and stores the consumed calorie data in the database 4 or the like (S12).

The state acquisition unit 32 calculates the user's upper limit calorie intake within a preset period based on the personal data, and stores the upper limit calorie intake data in the database 4 or the like (S13). The steps in S12 and S13 may be performed in reverse order.

Next, the control unit 33 of the processing apparatus 3 calculates the difference between the upper limit calorie intake and the consumed calorie. Based on the calculated difference and the ranking data for recommending food items to the user, the control unit 33 controls either the first drive mechanism 25 or the second drive mechanism 27 and also controls the conveying apparatus 23 (S14).

In more detail, for instance, the ranking data categorizes food items as follows: low-calorie food items (such as fish, tofu, etc.) are set as a rank A, high-calorie food items (such as meat, carbohydrates, alcoholic beverages, sweets, etc.) are set as a rank C, and other food items are set as a rank B.

If the difference between the upper limit calorie intake and the consumed calories is less than a preset first threshold, the control unit 33 of the processing apparatus 3 generates the recommendation data to recommend the user to consume the food items of the rank A.

Based on the recommendation data, the control unit 33 of the processing apparatus 3 controls either the first drive mechanism 25 or the second drive mechanism 27 and also controls the conveying apparatus 23 so that the boxes 22 containing the food items of the ranks B and C are arranged in the hard-to-reach areas of the storage unit main body 21, and the boxes 22 containing the food items of the rank A are arranged in the accessible areas of the storage unit main body 21.

When the difference between the upper limit calorie intake and the consumed calorie is greater than or equal to the preset first threshold and less than a second threshold that is greater than the first threshold, the control unit 33 of the processing apparatus 3 generates the recommendation data to recommend the user to consume the food items of the ranks A and B.

Then, based on the recommendation data, the control unit 33 of the processing apparatus 3 controls either the first drive mechanism 25 or the second drive mechanism 27 and also controls the conveying apparatus 23 so that the boxes 22 containing the food items of the rank C are arranged in the hard-to-reach areas of the storage unit main body 21, and the boxes 22 containing the food items of the rank A and B are arranged in the accessible areas of the storage unit main body 21.

Here, when the difference between the upper limit calorie intake and the consumed calorie is greater than or equal to the preset second threshold, the control unit 33 of the processing apparatus 3 may control either the first drive mechanism 25 or the second drive mechanism 27 and also control the conveying apparatus 23 so that, for example, the boxes 22 are arranged in the vacant areas A within the storage body 21 in the order of their arrival at the storage unit main body 21 without generating the recommendation data.

In this embodiment, the control unit 33 of the processing apparatus 3 determines that the difference between the upper limit calorie intake and the consumed calorie is less than the first threshold, and generates recommendation data to recommend the user to consume the food items of the rank A.

Then, the control unit 33 of the processing apparatus 3 controls the second drive mechanism 27 and also controls the conveying apparatus 23 so that second boxes 22b containing alcoholic beverages, sweets, meat, and carbohydrates, which are the food items of the rank C, are arranged in the first area A2-1, the second area A2-2, the fifth area A2-5, and the sixth area A2-6, which are the hard-to-reach areas of the refrigeration space S2 of the storage unit main body 21, and the second boxes 22b containing fish and tofu, which are food items of the rank A, are arranged in the third area A2-3 and the fourth area A2-4, which are the accessible areas of the refrigeration space S2 of the storage unit main body 21.

The second drive mechanism 27 opens the second door 26 on the rear side of the refrigeration space S2 of the storage unit main body 21. Next, the conveying apparatus 23 inserts the second boxes 22b containing alcoholic beverages, sweets, meat, and carbohydrates into the first area A2-1, the second area A2-2, the fifth area A2-5, and the sixth area A2-6 of the refrigeration space S2 of the storage unit main body 21, and inserts the second boxes 22b storing fish and tofu into the third area A2-3 and the fourth area A2-4 of the refrigeration space S2 of the storage unit main body 21. After that, the second drive mechanism 27 closes the second door 26 on the rear side of the refrigeration space S2 of the storage unit main body 21. Thus, the recommended food items for the user's intake can be arranged in the accessible areas of the storage unit main body 21.

Thus, the processing apparatus 3 and the processing method according to this embodiment arrange the recommended food items for the user's intake within the areas of the storage unit main body 21 that are easy for the user to access, that is, the accessible areas. Therefore, the user can be encouraged to actively pick up the recommended food items for him/her to consume.

### Second Embodiment

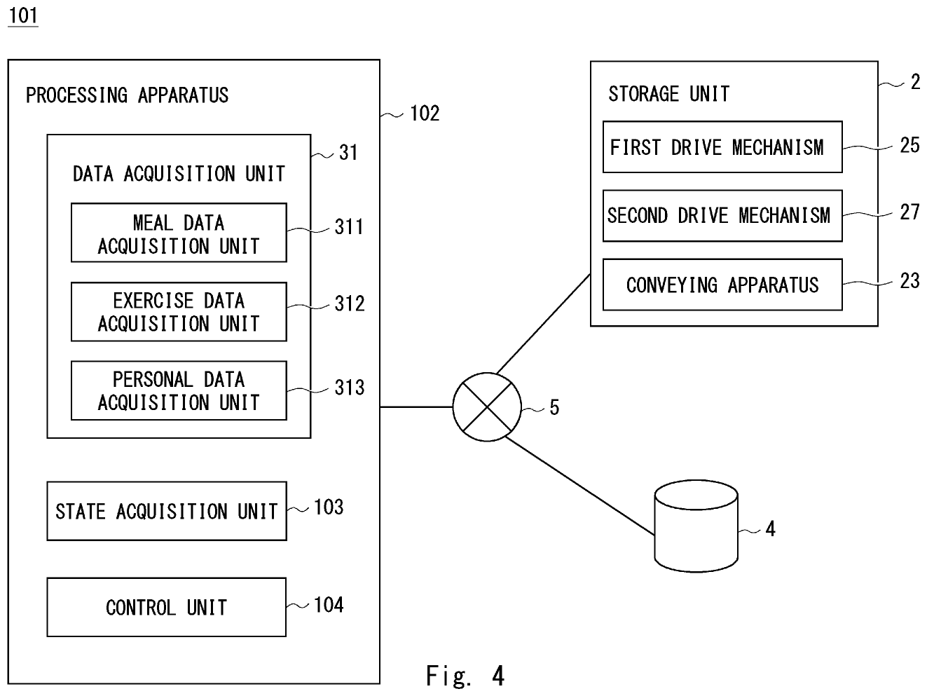

FIG. 4 is a block diagram showing a control system of a storage system according to this embodiment. Since a storage system 101 according to this embodiment has a configuration substantially equal to that of the storage system 1 according to the first embodiment as shown in FIG. 4, the repeated description will be omitted, and equivalent elements are described using the same reference signs.

In this embodiment, the state acquisition unit 103 of the processing apparatus 102 acquires health data of a user as lifestyle data, and obtains a blood sugar level or a stress state based on the health data. For example, using a wearable device such as a general smartwatch, the state acquisition unit 103 can acquire the user's blood sugar level or stress state.

The control unit 104 of the processing apparatus 102 generates recommendation data for food items to be consumed by the user based on the user's blood sugar level or stress state, controls either the first drive mechanism 25 or the second drive mechanism 27, and also controls conveying apparatus 23 based on the recommendation data.

At this time, for example, when the user's blood sugar level is low, food items rich in carbohydrates may be recommended, or when the user's stress state is high, foods rich in GABA (Gamma Amino Butyric Acid) may be recommended. Thus, according to the user's blood sugar level and stress state, the user can be encouraged to actively pick up the recommended food items for him/her to consume.

### Third Embodiment

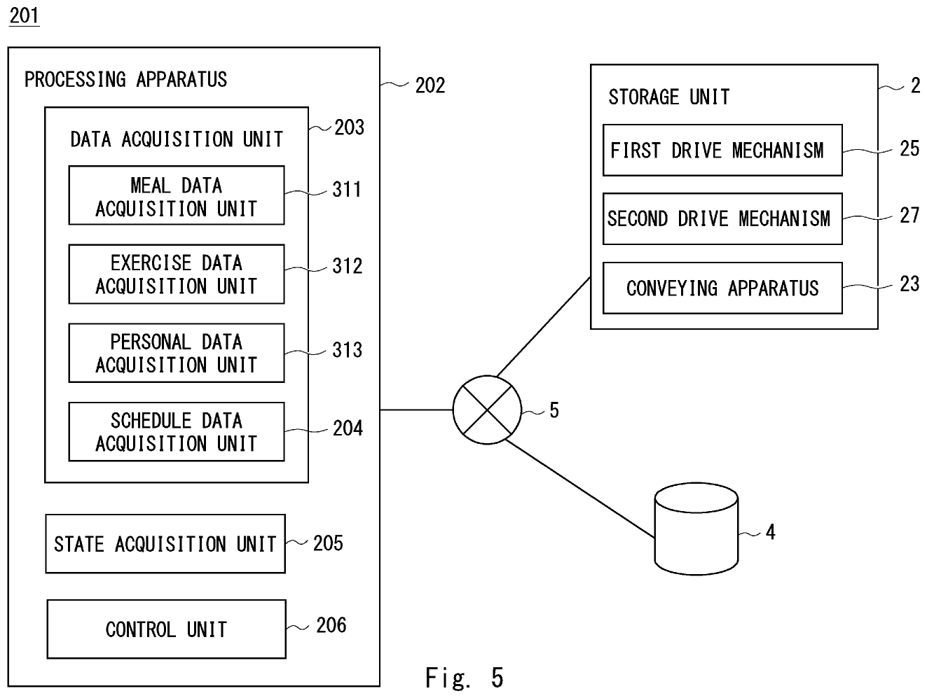

FIG. 5 is a block diagram showing a control system of a storage system according to this embodiment. Since a storage system 201 according to this embodiment has a configuration substantially equal to that of the storage system 1 according to the first embodiment as shown in FIG. 5, the repeated description will be omitted, and equivalent elements are described using the same reference signs.

As shown in FIG. 5, the processing apparatus 202 of the storage system 201 according to this embodiment includes a schedule data acquisition unit 204 in a data acquisition unit 203. The schedule data acquisition unit 204 acquires schedule data of a user as lifestyle data.

The schedule data includes, for example, information indicating the user's schedule such as work, tasks, desk work, meetings, commuting, travel, meals, dining, snacks, exercise, kindergarten pick-up, and package deliveries. For each planned item, there are designated start and end times in the schedule data. The schedule data acquisition unit 204 records the schedule data in the database 4 or the like.

The state acquisition unit 205 of the processing apparatus 202 acquires the user's snack time based on the schedule data. Next, the control unit 206 of the processing apparatus 202 controls either the first drive mechanism 25 or the second drive mechanism 27 and also controls the conveying apparatus 23 based on data indicating a snack time so that during the snack time, food items with low calories and good satiety are placed in the accessible areas of the storage unit main body 21. This makes it possible to restrain excessive calorie intake through snacks.

### Fourth Embodiment

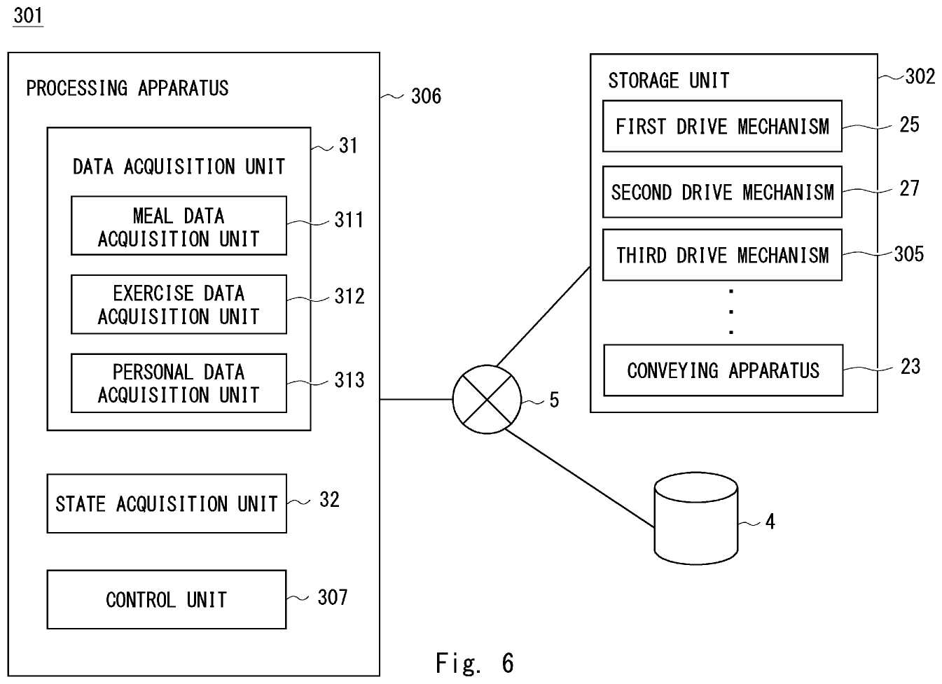

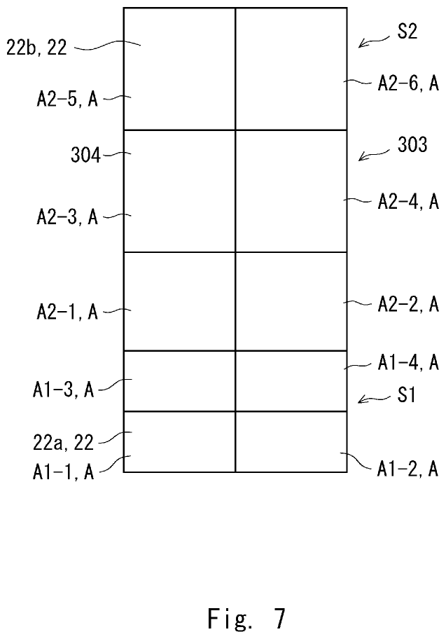

FIG. 6 is a block diagram showing a control system of a storage system according to this embodiment. FIG. 7 is a front view showing a storage unit main body and boxes of a storage unit according to this embodiment in a simplified manner. Since a storage system 301 according to this embodiment has a configuration substantially equal to that of the storage system 1 according to the first embodiment as shown in FIG. 6, the repeated description will be omitted, and equivalent elements are described using the same reference signs.

As shown in FIG. 7, the storage system 301 according to this embodiment has a configuration in which an openable and closable door 304 is provided on the front side of each area A of the storage unit main body 303 of the storage unit 302. The opening and closing of each door 304 is controlled by a third drive mechanism 305.

The control unit 307 of the processing apparatus 306 controls the third drive mechanism 305 so that the doors 304 of the areas (i.e., accessible areas) A, where the recommended food item for the user to consume are stored, can be opened and closed (i.e., the doors 304 are unlocked and capable of being opened), and the doors 304 of the other areas (i.e., hard-to-reach areas) A are locked in a closed state. Thus, it is possible to encourage the user to surely pick up the recommended food items for him/her to consume.

### Other Embodiment

For example, the non-recommendation data for food items that should not be recommended to the user may also be created based on the personal data in addition to the recommendation data. The food items that should not be recommended to the user may be actively arranged in the hard-to-reach areas. For example, in order to discourage the user who has a high purine level from picking up food items like fish roe, fish roe and the like may be arranged in the hard-to-reach areas of the storage unit main body.

For example, in the above embodiments, the accessible areas and the hard-to-reach areas are separated in the height direction of the storage unit main body. Alternatively, the accessible areas and the hard-to-reach areas may be separated in the depth direction of the storage unit main body. In this case, the accessible areas may be arranged on the front side of the storage unit main body.

For example, when a plurality of users are present, the users located in front of the storage unit main body may be specified based on image data from a camera provided in the storage unit and position data of each user acquired using a wearable device such as a smartwatch. At this time, it is preferable that the height of each area of the storage unit be configured to be changed according to the users' physique.

For instance, according to factors like time and user preferences, the conveying apparatus may be used to rearrange the arrangement of food items. For example, during the evening, alcoholic beverages may be moved to the accessible areas of the storage unit main body. In this case, when food items are replenished within the boxes 22, it can help prevent shortages.

Some or all of the above processing may be executed by a computer program. That is, the control of the above processing apparatus is executed when the control computer constituting the processing apparatus executes the program. The above program includes instructions (or software codes) that, when loaded into a computer, cause the computer to perform one or more of the functions described in the embodiment. The program may be stored in a non-transitory computer readable medium or a tangible storage medium. By way of example, and not a limitation, non-transitory computer readable media or tangible storage media can include a random-access memory (RAM), a read-only memory (ROM), a flash memory, a solid-state drive (SSD) or other types of memory technologies, a CD-ROM, a digital versatile disc (DVD), a Blu-ray disc or other types of optical disc storage, and magnetic cassettes, magnetic tape, magnetic disk storage or other types of magnetic storage devices. The program may be transmitted on a transitory computer readable medium or a communication medium. By way of example, and not a limitation, transitory computer readable media or communication media can include electrical, optical, acoustical, or other forms of propagated signals.

From the disclosure thus described, it will be obvious that the embodiments of the disclosure may be varied in many ways. Such variations are not to be regarded as a departure from the spirit and scope of the disclosure, and all such modifications as would be obvious to one skilled in the art are intended for inclusion within the scope of the following claims.

What is claimed is:

1. A processing apparatus comprising:
a data acquisition unit configured to acquire lifestyle data and personal data of a consumption requester of food items; and
a control unit configured to generate recommendation data for food items to be recommended for the consumption requester to consume based on the lifestyle data and the personal data, and control a storage unit so that the recommended food items stored in the storage unit becomes accessible by the consumption requester based on the recommendation data.

2. The processing apparatus according to claim 1, wherein the control unit generates the recommendation data based on data indicating a difference between an upper limit calorie intake of the consumption requester over a preset period calculated from the personal data and consumed calories over the preset period calculated from the lifestyle data.

3. The processing apparatus according to claim 1, wherein
the data acquisition unit acquires health data of the consumption requester as the lifestyle data, and
the control unit generates the recommendation data based on data indicating a blood sugar level or a stress state of the consumption requester acquired from the health data.

4. The processing apparatus according to claim 1, wherein
the data acquisition unit acquires schedule data of the consumption requester as the lifestyle data, and
the control unit generates the recommendation data based on data indicating a snack time estimated from the schedule data.

5. The processing apparatus according to claim 1, wherein the control unit controls the storage unit to unlock a locked state of an area containing the recommended food items among a plurality of locked areas containing food items within the storage unit based on the recommendation data.

6. The processing apparatus according to claim 1, wherein the control unit generates non-recommendation data for food items that are not recommended to be consumed by the consumption requester based on the personal data, and controls the storage unit based on non-recommendation data such that the food items that are not recommended are arranged in a preset hard-to-reach area within the storage unit.

7. The processing apparatus according to claim 6, wherein a preset accessible area for the food items within the storage unit is arranged on a front side of the storage unit in relation to the hard-to-reach area.

8. The processing apparatus according to claim 6, wherein the preset hard-to-reach area for the food items within the storage unit is arranged in a position of the storage unit that is higher or lower than that of the accessible area for the food items.

9. A storage system comprising:
the processing apparatus according to claim 1; and
a storage unit containing the food items.

10. A processing method comprising:
acquiring lifestyle data and personal data of a consumption requester of food items; and
generating recommendation data for food items to be recommended for the consumption requester to consume based on the lifestyle data and the personal data, and controlling a storage unit so that the recommended food items stored in the storage unit becomes accessible by the consumption requester based on the recommendation data.

11. A non-transitory computer readable medium storing a processing program for causing a computer to execute processing of:
acquiring lifestyle data and personal data of a consumption requester of food items; and
generating recommendation data for food items to be recommended for the consumption requester to consume based on the lifestyle data and the personal data, and controlling a storage unit so that the recommended food items stored in the storage unit becomes accessible by the consumption requester based on the recommendation data.
